(19) United States
(12) Patent Application Publication
(10) Pub. No.: US 2023/0106430 A1
(43) Pub. Date: Apr. 6, 2023

(54) Saliva Aspirator

(71) Applicant: Ivoclar Vivadent AG, Schaan (LI)

(72) Inventors: Markus Lichtensteiger, Montlingen (CH); Adrian Büchel, Planken (LI)

(21) Appl. No.: 17/934,040

(22) Filed: Sep. 21, 2022

(30) Foreign Application Priority Data
Sep. 23, 2021 (EP) 21198634.4

(51) Int. Cl. A61M 1/00; A61C 17/10

(52) U.S. Cl. CPC A61M 1/86; A61C 17/10

(57) ABSTRACT

A saliva aspirator having an injection moulded plastic component forming a coupling for connection to a suction hose, optionally via an adapter. A plastic moulded part is manufactured as a multi-component injection moulded part and forms a plastic component which is harder than another plastic component, the hard component, the coupling and optionally the adapter. The other plastic component, the soft or flexible component, forms a suction area with at least one suction opening or suction pore for saliva and an outlet opening towards the suction hose and consists in particular of an open-pored, preferably thermoplastic, elastomer.

### CROSS-REFERENCE TO RELATED APPLICATIONS

This application claims priority to European patent application No. 21198634.4 filed on Sep. 23, 2021, which disclosure is incorporated herein by reference in its entirety.

### TECHNICAL FIELD

The invention relates to a saliva aspirator.

### BACKGROUND

Saliva aspirators of this type have been known for a long time and are used to aspirate saliva and possibly other liquids from the mouth of a patient. Typically, such a saliva aspirator comprises a suction tube and a suction element. The suction tube is preferably curved and its curved section is hooked into the patient’s mouth.

If suction is to be applied in the oral region of the patient, the suction tube extends over the lower jaw tooth row and the suction element is then intended to develop the desired suction effect at the deepest point in the patient’s mouth.

The suction element is typically designed as a sponge and as such surrounds the suction tube at its oral end. If necessary, the suction tube is provided with small holes there, but in any case has an open end.

The sponge typically has to be manufactured separately. It is attached to the end of the suction hose. It is proportioned in such a way as to fit tightly there. This is to prevent it from accidentally slipping off the end of the suction tube in the patient’s mouth and then remaining as a foreign body in the patient’s mouth.

Typically, sponge-like bodies made of plastic do not have a long life and also lose their elasticity over time. Therefore, it may happen that such a sponge does detach from the suction tube when it has exceeded the recommended lifetime.

To prevent this, it has been suggested that small barbs be attached to the suction hose to prevent it from slipping off.

Gluing the sponge to the suction hose has also been suggested. However, the sponge can then no longer be detached from the suction hose and the suction hose has to be replaced frequently, which is uneconomical.

Sometimes it has been known to do without the sponge without further ado. This eliminates the problems associated with the sponge, but the comparatively hard suction hose is found to be uncomfortable, especially under the tongue.

Therefore, attempts have been made to combine the suction hose with a tongue holder that keeps the tongue away from both the teeth and the suction hose itself. However, this increases the pressure on the oral soft tissue through the opposite, lower end of the suction tube, which is also perceived as uncomfortable.

Solutions of this kind have been known for a long time, for example from DE 1 063 329 B1. Moreover, US 20160038348, 20160345815, 8231384, 20210038354 and 7335023 are directed to dental devices and are hereby incorporated by reference in their entirety.

Nowadays, such saliva aspirators are a mass-produced item and are manufactured and sold on a large scale. Therefore, costs also play a role.

### SUMMARY

Therefore, the invention is based on the task of creating a saliva aspirator, which is inexpensive to manufacture, safe to use and is perceived as agreeable by the patient.

A saliva aspirator according to the invention is characterised in that it has a special moulded plastic part. This is preferably attached to the end of the suction hose.

A moulded plastic part is typically dimensionally stable in itself and therefore generally hard. Such a moulded part would be perceived as uncomfortable by the patient.

According to the invention, the moulded part is manufactured as a multi-component injection moulded part. This measure has the surprising advantage of enabling a hard component and a soft component to be produced in one piece without incurring additional costs. The hard component can be used for coupling to the suction hose. The soft component may have at least one suction opening or suction pore. It may lie against the patient’s soft tissue, orally but also vestibularly, without being perceived as uncomfortable by the patient.

The patient’s saliva can be suctioned very efficiently through the soft component.

The hard component, on the other hand, is particularly suitable for connection to the suction tube. The firm fit ensures that the suction element cannot get lost. The hard component forms a coupling with which it can be coupled to the hose. The coupling is durable, at least in comparison with a sponge.

However, the plastic moulding can also be detached via the coupling if it needs to be specifically separated from the hose. This can be the case, for example, if the moulded part is to be disinfected or if it is to be replaced.

The moulded part can be of any suitable design.

In an advantageous embodiment, an adapter is provided which is connected in a non-detachable manner to the suction hose. In this embodiment, the coupling then couples to the adapter via releasable locking projections. This embodiment provides even greater security against loss of the moulded part.

According to the invention, it is in any case provided that the soft component forms a suction region. Preferably, the suction region has a plurality of suction pores. Preferably, the hard component has a shape which extends three-dimensionally in space and spans an area which is covered by the soft component, in particular its suction area. Accordingly, the soft component is not dimensionally stable, but is advantageously held in shape by the hard component.

Through multi-component injection moulding, the two components are very firmly and, effectively, joined together as one piece.

The production is carried out in a known manner in that one component, for example the harder one, is injected with one injection nozzle and the other component, for example the softer one, is injected with another injection nozzle. To this extent, 2 injection nozzles or injection units are used. However, only one closing unit is required.

The two components can thus be produced cost-effectively with only one mould and in one operation.

Preferably, a hollow chamber is provided by the hard component. The hollow chamber is elongated and has an outlet opening at one end and is closed at the other end. A large portion of the hollow chamber is occupied by the soft component, but preferably slightly less than half. The soft component extends along one longitudinal side of the hollow chamber.

The outlet opening is connected to the suction hose. Through it, the hollow chamber is put under negative pressure. The negative pressure causes saliva to be sucked in through the suction pores.

Preferably, the soft component is made of an open-pored elastomer and is quite soft so that it also adapts to the shape of the soft tissue in the patient’s mouth. The hard hollow chamber, on the other hand, enables a secure connection to the suction tube and prevents the suction area from collapsing.

The soft material is more flexible than the hard material and has a lower hardness. It is preferable that the soft material be in the range of 12 Shore A to 60 Shore A, 16 Shore A to 50 Shore A, or 30 Shore A to 40 Shore A. It is preferable that the soft material has an elongation at break up to about min 800 % and a tensile strength of approximately 4 - 10 MPa.

It is preferable that the hard material has a modulus of elasticity of from 1400 to 3200 MPa and an elongation at break up to about min 50%. Some examples include, but are not limited to, PEEK (polyether ether ketone), PC (polycarbonate), PET (polyethylene terephthalate), PP (polypropylene) PBT (poly butylene terephthalate), and nylon 66.

The hollow chamber preferably has a slightly curved design in its longitudinal direction. The soft component extends along the convex side of the hollow chamber so that it has a spherical shape there. This solution allows a good descent to the lowest point in the patient’s oral region.

In cross-section, the hollow chamber has an elliptical or oval shape. This makes it slim and less of a foreign body for the tongue.

The cross-section of the hollow chamber is considerably larger than the cross-section of the suction tube, for example about five to ten times as large. It is preferably constant over its length.

In an advantageous embodiment of the invention, the convex suction area has a profile structure with ribs or projections. The ribs or projections surprisingly increase the suction power of the saliva aspirator according to the invention. The suction pores are not closed by contact with the soft tissue.

In a further advantageous embodiment, a tongue retainer is provided which extends upwardly in one piece with the hard component, on the side thereof opposite the soft com ponent.

In a further preferred embodiment, the suction tube is provided with an integral wire retainer. This forms a thin channel within the suction hose. A wire is provided there, which is preferably engaged with the adapter when the suction element, i.e. the moulded plastic part, is connected to the suction hose.

It is preferable that the saliva aspirator is a plastic moulding made by injection moulding, having a coupling for connection to a suction hose, optionally an adapter for connection to the suction hose, wherein the plastic moulding is produced as a multi-component injection moulding comprising a hard and/or rigid plastic component and a soft and/or flexible plastic component, wherein the rigid plastic component has a hardness greater than the flexible plastic component and comprises the coupling, wherein the flexible plastic component comprises a suction region with at least one suction opening or pore for saliva and an outlet opening towards the suction hose.

It is preferable that the flexible plastic component has a hardness lower than the rigid plastic component and is fabricated of an open-pored, thermoplastic, elastomer.

It is preferable that the adapter is fabricated of the rigid plastic.

It is preferable that the plastic moulding comprises a tongue holder which extends away on a side of the moulding opposite the suction region.

It is preferable that the flexible plastic component is dimensionally stable with respect to the suction vacuum of the saliva aspirator, at a level of between 80 mbar and 250 mbar.

It is preferable that the rigid component extends at least partially or completely around the outlet opening and keeps the outlet opening stretched open.

It is preferable that the suction region is formed by a hollow chamber which is closed on all sides apart from the at least one suction opening or pore and the outlet opening, and which suction region is under negative pressure during operation of the saliva aspirator.

It is preferable that the suction region, on a side opposite the tongue holder, is formed entirely by the flexible component.

It is preferable that the rigid component and the flexible component are firmly connected to each other by bonded injection moulding.

It is preferable that the outlet opening has a cross-section which is larger than a cross-section of the suction tube and, in particular, a cross-section which is larger than a circular cross-section in the direction of the tongue holder.

It is preferable that the tongue holder extends in extension of a highly oval or elliptical cross-section of the suction area.

It is preferable that the suction region has a cross-section which is substantially constant over its longitudinal extension up to the outlet opening, which cross-section is larger than a free flow cross-section of the suction hose.

It is preferable that the suction region has, on a side opposite the tongue holder or the rigid component, a structure profile with ribs or projections, with a profile height which is substantially the same over a course of the side.

It is preferable that the structure profile has a height of more than 0.2 mm or about 0.5 mm, and wherein the suction pores of the suction region end at a base of the profile structure, such that, when the structure profile bears against tissue, the structure profile prevents the suction pore from also blocking the tissue.

It is preferable that the adapter can be releasably coupled to the coupling of the moulded part and, wherein the adapter produces a cross-sectional adaptation between the suction tube and the outlet opening and is made of the rigid plastic as a part separate from the moulded part.

It is preferable that the adapter is configured for positive connection to the plastic moulded part and/or is positively connected to the suction hose via the coupling.

It is preferable that the suction hose has an integrated wire receptacle for receiving a plastically deformable wire, which ends at the adapter and/or at the plastic moulding for connecting the wire thereto.

It is preferable that the wire extends into the adapter and is captively anchored therein, such that the suction hose cannot be separated from the adapter without being destroyed.

### BRIEF DESCRIPTION OF THE DRAWINGS

Further advantageous features and details will be apparent from the following description of an embodiment of the invention with reference to the drawings.

FIG. 1 shows a schematic view of a saliva aspirator according to the invention in fully assembled form;

FIG. 2 shows the embodiments according to FIG. 1, but in separate form;

FIG. 3 shows an adapter for a saliva aspirator according to the invention;

FIG. 4 shows a schematic perspective view of a moulded plastic part for a saliva aspirator according to the invention;

FIG. 5 shows a sectional view through the plastic moulding according to FIG. 4;

FIG. 6 shows a perspective view of the saliva aspirator according to the invention showing the end of the suction tube; and

FIG. 7 shows an enlarged view of the end of the suction hose.

### DETAILED DESCRIPTION

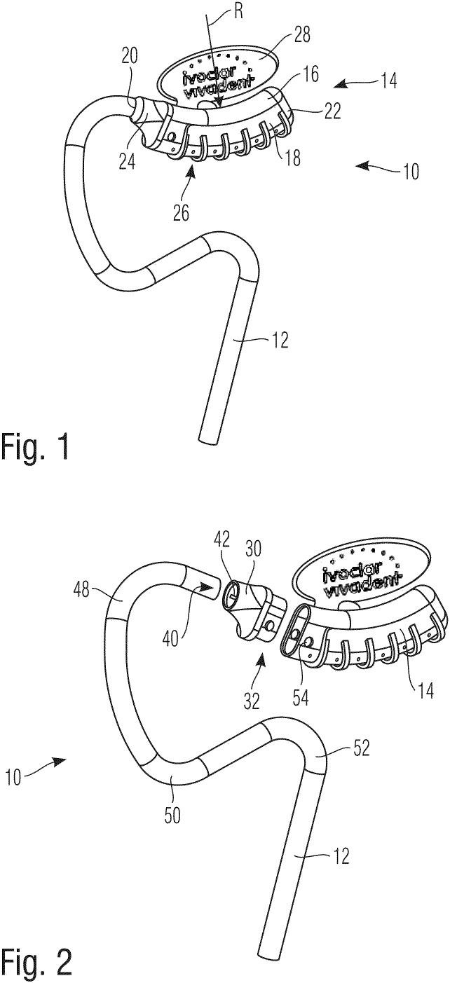

In FIG. 1, a saliva aspirator 10 is schematically shown. The saliva aspirator 10 comprises a suction hose 12 and a plastic moulding 14 which is injection moulded, i.e., produced by injection moulding.

The moulded part 14 is designed as a multi-component injection moulded part. It comprises a hard plastic component 16 and a softer plastic component 18, both of which are produced in a single operation and are integrally joined together. The harder plastic component 16 can be any suitable biocompatible plastic which has sufficient properties for the intended application. The softer plastic component is a plastic that has pores or small openings suitable for the passage of liquids.

The moulding 14 is placed under negative pressure by the suction hose 12. This causes the pores or openings in the softer plastic component to have a suction function.

For example, the softer plastic component may be an open pore elastomer. It is also possible to use a softer plastic component that has micro-tubes extending towards the interior of the moulding 14. The softer component may also be in the form of a sponge. Furthermore, it may also have a filtering function.

As can be seen from FIG. 1, the moulding 14 is slightly curved, and has a radius of curvature extending transversely to its longitudinal direction. The moulding 14 is formed as a hollow chamber. It extends longitudinally in the manner of a cylinder, which is however curved. The ratio between the diameter of this curved cylinder and the length is between 1 to 2 and 1 to 10, and in the illustrated embodiment example about 1 to 4.

The harder plastic component 16 occupies most of the outer wall of the hollow chamber 14. It also extends over one end of the moulding 14, which forms an outlet opening 20, and an opposite end 12, which is closed.

A coupling 24 is formed at the outlet opening 20 for connection to the suction hose 12.

Between these two ends, but only on one side of the curved cylinder of the moulding or hollow chamber 14, the softer plastic component 18 is formed. It also provides a suction area 26. This has the suction tubes already mentioned and is used to aspirate saliva. There is a negative pressure inside the hollow chamber 14, which is generated by the suction tube 12.

The curved cylindrical hollow chamber 14 has a convex area where the softer component is located and a concave area where the harder plastic component 16 is located. The cross-section of the hollow chamber 14 is substantially elliptical. When inserted into the patient’s mouth, the softer plastic component 18 extends downward and the harder plastic component extends upward. In this respect, the cross-section of the moulding 14 can also be described as highly elliptical or highly oval.

A tongue holder 28 is arranged on the side opposite the softer plastic moulding 18. This is also integrally connected to the moulding 14 and extends upwards when the saliva aspirator 10 is inserted into the patient’s mouth. Its purpose is to keep the tongue away from the teeth.

By softly supporting the moulding 14 via the soft component 18 against the oral soft tissue, the tongue guard is held upwardly in an upright position without causing pain or irritation to the patient and protects the teeth being treated from the activities of the tongue.

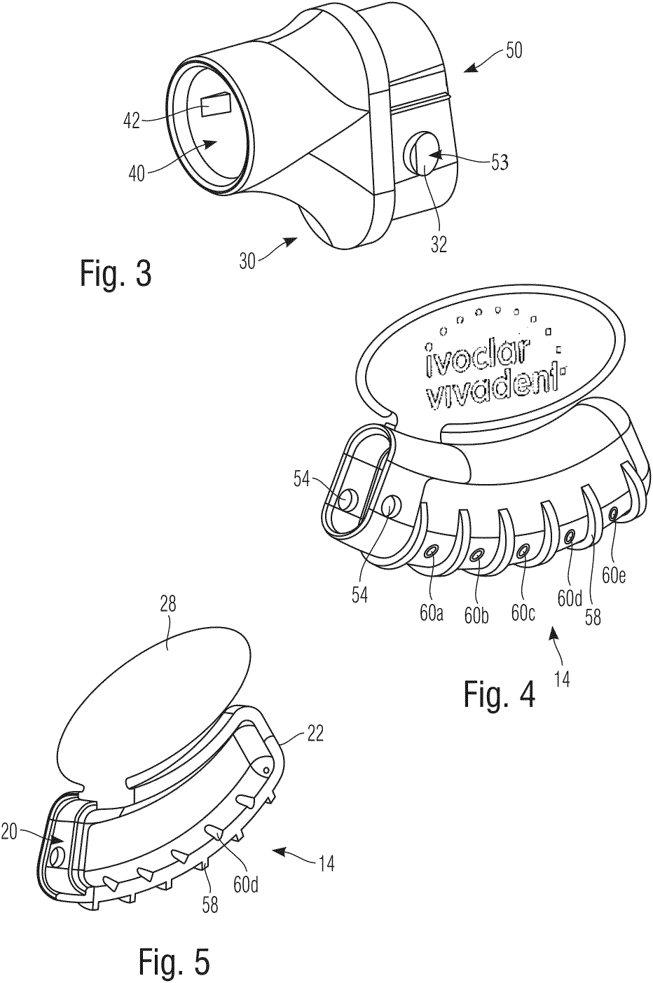

The suction tube 12 is removably inserted into the coupling 24. As can be seen from FIG. 3, the coupling 24 has special measures to prevent the suction hose from accidentally coming loose.

FIG. 2 shows the parts of the saliva aspirator according to the invention in detail.

It can be seen that the coupling 24 is formed on a separate moulding 30, which also constitutes an adapter. The adapter 30 is also formed of hard plastic, corresponding to the harder plastic component 16, and is connected to the moulded part 14 via locking elements 32.

The adapter 30 also has inward-facing locking tongues 42 on its reception opening 40, which is intended to receive the suction hose 12. These are used for positive engagement with the outside of the comparatively soft suction hose 12. With this specially designed receiving opening 40, the adapter 30 is, in effect, captively connected to the suction hose 12.

If the moulded part 14 needs to be replaced, it is preferably uncoupled from the adapter 30 by releasing the latching device 32. The adapter 30 therefore remains on the suction hose 12, and the moulding 14, which may need to be disinfected or replaced, can be dealt with according to the desired purpose.

The same or a new moulded part 14 can then be used.

As can be seen in FIGS. 1 and 2, the suction hose 12 has several curves. It has a dental arch 48. At this point, the suction tube 12 crosses the patient’s row of teeth or, if necessary, an area distal to the patient’s rearmost molar.

Starting from the dental arch 48, the suction tube 12 — shown somewhat shortened —traverses the patient’s lower lip and then runs downwards from there. At a chin arch 50 it is bent distally, i.e., towards the patient’s neck, and then again in the opposite direction at a neck arch 52.

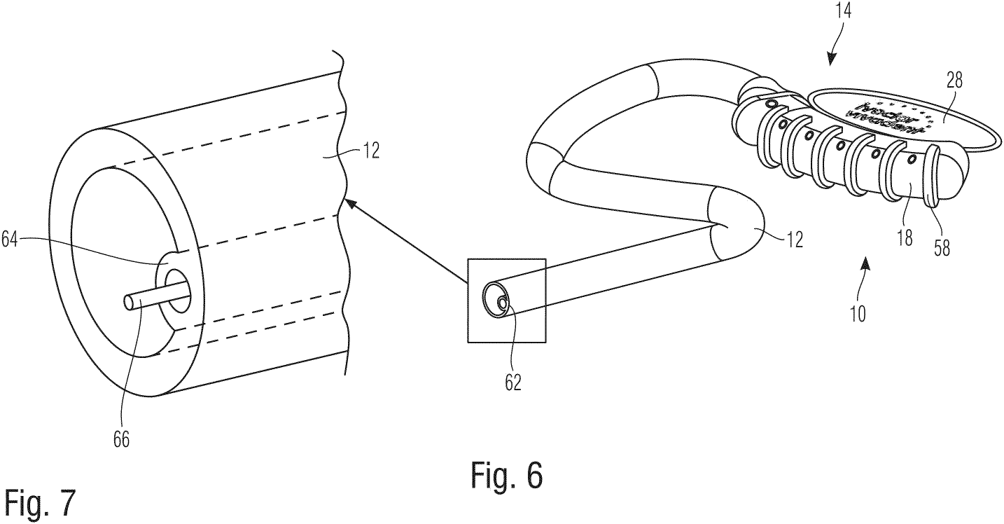

This design ensures that the moulding 14 is pressed downwards in the patient’s mouth, i.e., either vestibularly downwards or in the oral region downwards. The pressure is applied either by the preformed suction tube 12 itself or by a wire which is preformed and elastic and is described in more detail in FIGS. 6 and 7.

FIG. 3 shows the adapter 30 in detail. Identical reference signs here as well as in the other Figs. indicate identical or corresponding parts. FIG. 3 shows the highly oval design of the end 50 of the adapter 30 on the moulded part side. The receiving opening 40 has the aforementioned locking lugs on the side, of which the locking lug 42 can be seen in FIG. 3.

The latching device 32 is formed by a latching projection 53 and an opening 54 in the moulded part 14 shown in FIG. 4, which is suitably designed for this purpose. The latching projection 53 enters the latching recess 54 and passes through it. By pressing on the latching projection 53, the adapter 30 can be uncoupled from the moulding 14.

The receiving opening 40 is dimensioned such that the suction hose 12 can be inserted into it with a form fit. Preferably, the suction hose 12 is briefly notched at suitable points in order to be able to receive the locking tongues 42 in a form-fitting manner. Then the connection is so tight that it will not loosen even under normal pulling.

FIG. 4 shows an enlarged perspective view of a moulded part 14. In the region of the soft component 18, the moulding 14 has downwardly projecting ribs 58. Between these ribs numerous suction pores extend between them, of which suction pores 60a to 60e can be seen in FIG. 4. The suction pores 60a to 60e spring back upwards in relation to the ribs 58. This prevents the soft tissue located there from covering the pore openings.

FIG. 5 shows a sectional view of the moulding 14. The section is also provided through the tongue retainer 28, which accordingly shows no labelling. It can be seen that the moulding 14 is formed as a hollow chamber 22.

FIG. 6 shows a somewhat modified embodiment of the saliva aspirator 10 according to the invention. The difference relates to the design of an integrated wire holder 62, which comprises a channel 64 which is more clearly visible in FIG. 7. The channel 64 is formed integrally with the suction hose 12 and extends at its outer edge, with a diameter which is significantly smaller than the diameter of the suction hose 12, for example one fifth to 1/20 of the diameter of the suction hose 12.

A wire 66 can be inserted into the channel 64. The wire 66 is basically flexible, but has a certain elasticity. This means that it can press the suction tube against the patient’s chin and thereby press the moulding 14 downwards.

The wire 66 can also be used to better anchor the adapter 30 to the suction hose 12. For this purpose, it is inserted into the adapter 30 in a corresponding opening and then bent off here. This creates a positive fit between the wire 66 and thus the suction hose 12 and the adapter 30.

In this respect, the wire 66 can fulfil a dual function.

The terms “about” and “substantially” are intended to include the degree of error or uncertainty associated with measurement of the particular quantity or shape as one of ordinary skill in the art would understand.

Elastic and soft have been used to describe the soft component and hard and rigid have been used to describe the hard component.

1. A saliva aspirator comprising
a plastic moulding made of injection moulding, comprising a coupling for connection to a suction hose,
optionally an adapter for connection to the suction hose,
wherein the plastic moulding is produced as a multi-component injection moulding comprising a rigid plastic component and a flexible plastic component,
wherein the rigid plastic component has a hardness greater than the flexible plastic component and comprises the coupling,
wherein the flexible plastic component comprises a suction region with at least one suction opening or pore for saliva and an outlet opening towards the suction hose.

2. The saliva aspirator according to claim 1,
wherein the flexible plastic component has a hardness lower than the rigid plastic component and is fabricated of an open-pored, thermoplastic, elastomer.

3. The saliva aspirator according to claim 1,
wherein the adapter is fabricated of the rigid plastic.

4. The saliva aspirator according to claim 1,
wherein the plastic moulding comprises a tongue holder which extends away on a side of the moulding opposite the suction region.

5. The saliva aspirator according to claim 1,
wherein the flexible plastic component is dimensionally stable with respect to the suction vacuum of the saliva aspirator, at a level of between 80 mbar and 250 mbar.

6. The saliva aspirator according to claim 1,
wherein the rigid component extends at least partially or completely around the outlet opening and keeps the outlet opening stretched open.

7. The saliva aspirator according to claim 1,
wherein the suction region is formed by a hollow chamber which is closed on all sides apart from the at least one suction opening or pore and the outlet opening, and which suction region is under negative pressure during operation of the saliva aspirator.

8. The saliva aspirator according to claim 1,
wherein the suction region, on a side opposite the tongue holder, is formed entirely by the flexible component.

9. The saliva aspirator according to claim 1,
wherein the rigid component and the flexible component are firmly connected to each other by bonded injection moulding.

10. The saliva aspirator according to claim 1,
wherein the outlet opening has a cross-section which is larger than a cross-section of the suction tube.

11. The saliva aspirator according to claim 1,
wherein the tongue holder extends in extension of a highly oval or elliptical cross-section of the suction area.

12. The saliva aspirator according to claim 1,
wherein the suction region has a cross-section which is substantially constant over its longitudinal extension up to the outlet opening, which cross-section is larger than a free flow cross-section of the suction hose.

13. The saliva aspirator according to claim 1,
wherein the suction region has, on a side opposite the tongue holder or the rigid component, a structure profile with ribs or projections, with a profile height which is substantially the same over a course of the side.

14. The saliva aspirator according to claim 13,
wherein the structure profile has a height of more than 0.2 mm or about 0.5 mm, and
wherein the suction pores of the suction region end at a base of the structure profile, such that, when the structure profile bears against tissue, the structure profile prevents the suction pore from also blocking the tissue.

15. The saliva aspirator according to claim 1,
wherein the adapter can be releasably coupled to the coupling of the moulded part and,
wherein the adapter produces a cross-sectional adaptation between the suction tube and the outlet opening and is made of the rigid plastic as a part separate from the moulded part.

16. The saliva aspirator according to claim 1,
wherein the adapter is configured for positive connection to the plastic moulding and/or is positively connected to the suction hose via the coupling.

17. The saliva aspirator according to claim 1,
wherein the suction hose has an integrated wire receptacle for receiving a plastically deformable wire, which ends at the adapter and/or at the plastic moulding for connecting the wire thereto.

18. The saliva aspirator according to claim 1,
wherein the wire extends into the adapter and is captively anchored therein, such that the suction hose cannot be separated from the adapter without being destroyed.
